(19) United States
(12) Patent Application Publication
(10) Pub. No.: US 2008/0051667 A1
(43) Pub. Date: Feb. 28, 2008

(54) Method And Device For Measuring Physiological Parameters At The Hand

(76) Inventor: Rami GOLDREICH, (US)

Correspondence Address: SMITH FROHWEIN TEMPEL GREENLEE BLAHA, LLC, Two Ravinia Drive, Suite 700, ATLANTA, GA, 30346, US

(21) Appl. No.: 11/571,848

(22) PCT Filed: May 16, 2004
(86) PCT No.: PCT/IL04/00417

§ 371 Date: Jan. 9, 2007

(51) Int. Cl. A61B 5/02

(52) U.S. Cl. 600/481

(57) ABSTRACT

A wrist-mounted device (100) for measuring at least one physiological parameter of the user. The present invention enables such a measurement to preferably be transformed into clinically useful information about the user. Such information may then optionally be sent to medical personnel, for example at a contact and/or monitoring center. The measuring parameters may include blood pressure, ECG, location. The present invention can perform a Holter process over more than one physiological parameter.

### FIELD OF THE INVENTION

The present invention is of a method and device for measuring at least one physiological parameter of a subject at the wrist, preferably for extracting clinically useful information thereof. More specifically, the present invention is of a device which may be worn at the wrist of the subject with a strap or other fastening article, and which may then be used to monitor the subject through measurement of the physiological parameter.

### BACKGROUND OF THE INVENTION

Currently, a number of different types of devices are available for monitoring human subjects in a non-invasive manner. For example, heart function can be monitored in a user through the use of electrodes, which must be attached to the skin of the user. Such equipment is very expensive, limiting its use to hospitals and other medical settings in which both the cost and the discomfort of the patient can be justified. Furthermore, patients may become anxious when examined by medical personnel, thereby significantly altering the normal readings for these patients.

However, there are many different situations in which non-invasive monitoring of a human subject is desired. For example, such monitoring could be very useful as part of the overall health maintenance of the human subject, and could be used in order to detect deterioration of the physiological condition of the subject before a concomitant deterioration in the health of the subject becomes noticeable. Examples of adverse physiological conditions which could be detected with regular non-invasive monitoring include but are not limited to excessive weight gain or loss; arrhythmia and other heart conditions; incipient diabetes in the form of improper glucose metabolism; and loss of lung capacity or other problems with respiration.

Heart rate, Breathing rate, body temperature, oxygen level in the blood and blood pressure are important factors in determining the state of a person's health and the physical condition of a person's body especially if exposed to physical or emotional stress. Periodic monitoring of these physical parameters is particularly important for individuals having cardiac disease and/or lowered cardiac functioning or high blood pressure. However, physically healthy individuals may also wish to periodically monitor their heart rate and blood pressure in order to monitor changes in their personal vital signs.

In order to support regular monitoring of human subjects in their normal environment, such as in the home and at the office for example, the equipment must be non-invasive and easy to use. The equipment would then be able to monitor at least one physiological parameter of the user, without requiring the user to perform any complicated actions and/or to operate complex devices. Indeed, it would be highly preferred for the equipment to be incorporated as part of the regular daily living routine of the subject, since the requirement for any additional or special actions on the part of human subject is likely to result in decreased compliance. In addition, the equipment should be robust yet inexpensive.

One example of such a device incorporates a wristband to attach a physiological sensor to the wrist of the subject. Currently, a number of different types of such wristband devices are available, most of which are intended to be used as stand-alone devices to provide information about the subject's own physical condition, mainly for heart rate and blood pressure. Most of these devices obtain such measurements by using an inflating cuff, which is bulky and awkward for the subject.

Wrist-mounted heart rate monitors are known to the art and have been disclosed, for example, in the patent to Orr et al, U.S. Pat. No. 3,807,388, wherein the duration of a heart beat is measured by counting electrical pulses recurring at a known frequency. The duration of the heartbeat is then related to a particular average heart beat rate. However, the disclosed measurement system does not directly measure the heart rate and, therefore, is subject to inaccuracies of measurement due to the instability of heart beat duration over brief intervals of time.

A blood pressure measuring device is disclosed in the patent to Petzke et al, U.S. Pat. No. 3,926,179, in which a probe is applied adjacent to the radial artery of a wrist. A pressure-sensitive transducer on the probe generates electrical signals corresponding to the blood pressure pulses of the radial artery. The electrical pulses are applied to analog circuitry that generates a systolic signal corresponding to the integrated voltage at the peak of the electrical pulse signal and a diastolic signal corresponding to the voltage at the low point of the pulse signal. The analog device of Petzke et al requires a substantial amount of power to operate and, therefore, is not suitable for use in a small, compact stand-alone device for being worn on the wrist.

A blood pressure and a heart rate measuring wrist watch is also disclosed in the patent to Broadwater, U.S. Pat. No. 4,331,154, in which a digital watch is employed to measure systolic and diastolic blood pressure as well as heart rate. The band of the watch supports a piezoelectric transducer that is held in contact with the wrist adjacent to the radial artery when a switch on the band is activated. The absolute values required for this method to evaluate blood pressure cause the device to be subject to inaccurate readings, since the tissues of the hand and wrist may be expected to expand and contract according to such factors as the time of day, and the condition of the external environment such as the atmospheric pressure. Such expansion or contraction may cause different degrees of tension on the wrist-mounted device, which is therefore not suitable for use without daily calibrations.

Other wrist-mounted devices are for wireless panic alarm systems, mainly for elderly people who live alone. These devices are usually shaped as a wristband or a pendant. Whenever the user becomes distressed, the user presses a panic button located on the device. The device then sends a digitally coded wireless message to a gateway device located nearby, usually in the same room, by using a unidirectional wireless data communication link. The gateway device then contacts a manually operated contact center, for example with a land based or cellular telephone connection. A particular identifier for the user is usually sent first, after which the human operator is allowed to talk to the user through a speaker and to listen through a sensitive microphone located within the gateway. However, none of the above systems contains any physiological measurement device within, in order to learn about the current physiological status of the user.

In such a situation as described above, the operator at the call center learns about the user's condition only by speaking with the user. However, this is only possible if the user is actually able to speak. High levels of background noise may also prevent the user from being heard by the microphone of the gateway device.

### SUMMARY OF THE INVENTION

The background art does not teach or suggest a device which can conveniently, non-intrusively and autonomously measure one or more physiological parameters, in order to extract medical information such as heart rate, breathing rate and blood pressure, and which may be worn on the wrist of the user. The background art also does not teach or suggest such a wrist-mounted device, which can measure such parameters and then send the information to a secured, automated databank (contact center) or call center containing medical personnel. The background art also does not teach or suggest such a wrist-mounted device which is compact, non-invasive, and light.

The present invention overcomes these deficiencies of the background art by providing a wrist-mounted device for measuring at least one physiological parameter of the user. The present invention enables such a measurement to preferably be transformed into medical information about the user, and/or displays the results on a LCD display. As used herein, the term “physiological parameter” refers to the signal which is received from the sensor, while the term “medical information” refers to the information which may be extracted or otherwise obtained by analyzing this signal and/or a combination of signals. Such information may then optionally be sent to medical personnel (for example at a contact monitoring call center) and/or to a remote secured, automated databank server usually Web based, through a gateway device. The gateway device preferably communicates with the wrist-mounted device of the present invention through a wireless communication channel.

The present invention has the option to display the medical information to the user on a local display, such that the user is optionally and preferably able to read the result locally. Examples of medical information which may be extracted from the measured physiological parameter or parameters include, but are not limited to: heart rate; variability in heart rate; breathing rate; arrhythmia of the heart (if any), as well as the general rhythm and functioning of the heart; blood pressure; presence of abnormal body movements such as convulsions for example; body position; general body movements; body temperature; presence and level of sweat; oxygen pressure in the blood; and glucose levels in the blood.

Optionally and more preferably, the present invention also features an alarm signal for being transmitted through the gateway device in order to indicate an emergency or otherwise dangerous situation for the user. The alarm signal may optionally be transmitted according to a manual action of the user, such as pressing a “panic button” for example.

Upon receipt of the manually activated alarm signal, the gateway would preferably initiate immediately a call to a human operated call center. Then the device would preferably automatically collect one or more current measurements of physiological parameters of the user. These measurements may be sent directly to the gateway, or alternatively may be analyzed in order to compute the medical information of the user before sending the results to the gateway. The human operator would then preferably be able to assess the user's medical condition from the received information.

Most preferably, the alarm signal is transmitted automatically upon measurement of one or more physiological parameters of the user, even if the user is unable to press the panic button. Optionally, the alarm signal may be given to the user, additionally or alternatively, for example by sounding an audible alarm, more preferably from the wrist-mounted device itself.

The device of the present invention also monitors, at least periodically or continuously, one or more physiological parameters of the user. Continuous monitoring would more easily enable the device to transmit the alarm signal if one or more physiological parameters are determined to be above predefined criteria, which may represent such medical information as unstable or excessive heart rate, or very high or low blood pressure.

According to an exemplary embodiment of the present invention, the wrist-mounted device features one or more sensors attached to a wristband or other fastening article. The sensor(s) may optionally be connected to a microprocessor, optionally by a wire but alternatively through a wireless connection. The microprocessor may optionally also be located within the wristband, or otherwise attached to the wristband. The sensor(s) may optionally support automatic collection of the measurement of the at least one physiological parameter, while the microprocessor is able to execute one or more instructions for extracting medical information about the user from such measurement(s).

The microprocessor more preferably operates a software program to process and analyze the data which is collected, in order to compute medical information. The extracted information, optionally also with the raw data, is then preferably transferred to the previously described gateway device. The gateway device may optionally relay such information to a remote server, which more preferably is able to provide such information to medical personnel, for example as part of a contact center. Therefore, continuous monitoring of the medical information and/or physiological parameters of the user may optionally and more preferably be made, enabling better medical care for the user. According to the present invention there is provided a device for measuring at least one physiological parameter of a subject, comprising: (a) a fastening article for being fastened to a wrist of the user; (b) a sensor for measuring at least one physiological function of the user, the sensor being in contact with at least a portion of the wrist and the sensor being attached to the fastening article; and (c) a processor for receiving a signal from the sensor and for converting at least one measurement to form the at least one physiological parameter. Optionally, the data may be stored on a non-volatile memory for being downloaded later by the user or by an operator.

According to another embodiment of the present invention, there is provided a system for measuring at least one physiological parameter of a subject, comprising: (a) a device for measuring the at least one physiological parameter, comprising: (i) a fastening article for being fastened to a wrist of the user; (ii) a sensor for measuring at least one physiological parameter of the user, the sensor being in contact with at least a portion of the wrist and the sensor being attached to the fastening article; (iii) a communication unit for at least transmitting data; and (b) a gateway device for receiving the transmitted data for being monitored.

According to another embodiment of the present invention, there is provided a method for monitoring a physiological parameter of a user, comprising: providing a device for monitoring the physiological parameter, the device being attached to at least a portion of the user at a pulse point of the user; monitoring the physiological parameter through the pulse point; and if a level of the physiological parameter of the user is outside of an expected range, transmitting an alarm.

According to still another embodiment of the present invention, there is provided a device for measuring at least one physiological parameter of a subject, comprising: (a) a fastening article for being fastened to a wrist of the user; (b) a piezoceramic sensor for measuring at least one physiological parameter of the user at a pulse point of the wrist and the sensor being attached to the fastening article; and (c) a processor for receiving a signal from the sensor and for converting the at least one measurement to form medical information.

Hereinafter, the term “microprocessor” includes, but is not limited to, general-purpose microprocessor, a DSP, a micro-controller or a special ASIC designed for that purpose.

Hereinafter, the term “wrist” includes, but is not limited to, the lower forearm from the elbow to the hand, inclusive, unless otherwise noted.

The method of the present invention could be described as a process for being performed by a data processor, and as such could optionally be implemented as software, hardware or firmware, or a combination thereof. For the present invention, a software application could be written in substantially any suitable programming language, which could easily be selected by one of ordinary skill in the art. The programming language chosen should be compatible with the computational device (computer hardware and operating system) according to which the software application is executed. Examples of suitable programming languages include, but are not limited to, Visual Basic, Assembler, Visual C, standard C, C++ and Java.

### BRIEF DESCRIPTION OF THE DRAWINGS

The invention is herein described, by way of example only, with reference to the accompanying drawings, wherein:

FIG. 1 is a schematic block diagram of a system according to the present invention;

FIG. 2 shows an exploded view of the device;

FIG. 3 describes a general state flow diagram;

FIG. 4 describes a bi-directional message format between the device and the gateway;

FIG. 5 shows an exploded view of an exemplary device with ECG option;

FIG. 6 shows an exploded view of an exemplary device, which illustrates the installation of a SpO2 sensor;

FIGS. 7A-7D show diagrams of external views of different parts of the illustrative blood pressure monitoring device according to the present invention;

FIG. 8 shows a schematic block diagram of a system according to the present invention featuring the blood pressure monitoring device of FIG. 7;

FIG. 9 is a flowchart of an exemplary method according to the present invention for performing an extended cardiac monitoring task; and

FIG. 10 is a flowchart of an exemplary method according to the present invention for synchronizing a medical care management function of the device according to the present invention with a central care facility.

### DESCRIPTION OF THE PREFERRED EMBODIMENTS

The present invention is of a wrist-mounted device for measuring at least one physiological parameter of the user. The present invention enables such a measurement to preferably b e transformed into medical information about the user. Such information may then optionally be sent to medical personnel (for example at a contact monitoring center) and/or to a remote server, through a gateway device. The gateway device preferably communicates with the wrist-mounted device of the present invention through a wireless communication channel.

Examples of medical information which may be extracted from the measured physiological parameter or parameters include, but are not limited to: heart rate; regularity in heart rate; breathing rate; I/E ratio; arrhythmia of the heart (if any), as well as the general rhythm and functioning of the heart; blood pressure; presence of abnormal body movements such as convulsions for example; body position; general body movements; body temperature; presence and level of sweat; oxygen saturation in the blood; and glucose levels in the blood.

Optionally and more preferably, the present invention also features an alarm signal for being transmitted through the gateway device in order to indicate an emergency or otherwise dangerous situation for the user. The alarm signal may optionally be transmitted according to a manual action of the user, such as pressing a “panic button” for example.

Most preferably, the alarm signal is transmitted automatically upon measurement of the one or more physiological parameters of the user, preferably even if the user is unable to press the panic button. Optionally, the alarm signal may be given to the user, additionally or alternatively, for example by sounding an audible alarm, more preferably from the wrist-mounted device itself.

An exemplary embodiment of the present invention may measure also parameters that may affect the subject's physical condition, including but not limited to ambient temperature and humidity, lighting conditions, smoke and/or other material in the air, distance from home etc.

Upon receipt of the manually/automatically activated alarm signal, the gateway would preferably initiate immediately a call to a human operated call center. Then the device would preferably automatically collect one or more current physiological measurements of the user. These measurements may be sent directly to the gateway, or alternatively may be analyzed in order to compute the medical parameters of the user before sending the results to the gateway. The gateway may also analyze the measurement, for example when the measurements are transferred directly to the gateway. The human operator, at the medical center, would then preferably be able to assess the user's medical condition from the received information. It should be noted that the terms “medical center” and “call center” are used interchangeably herein.

The device of the present invention may also monitor, at least periodically but more preferably continuously, the value or condition of one or more physiological parameters of the user. Continuous monitoring would more easily enable the device to transmit the alarm signal if measurements of one or more physiological parameters are collected and analyzed by the microprocessor to form medical information, which then could be determined to be above predefined criteria, such as unstable heart rate, or very high or low blood pressure, for example.

According to a non-limiting exemplary embodiment of the present invention, the wrist-mounted device features one or more sensors attached to a wristband or other fastening article. The sensor(s) are preferably connected to a microprocessor, optionally by a wire but alternatively through a wireless connection. The microprocessor may optionally also be located within the wristband, or otherwise attached to the wristband. The sensor(s) preferably support automatic collection of at least one physiological measurement; more preferably, the microprocessor is able to execute one or more instructions for extracting clinically useful information about the user from such measurement(s).

The microprocessor more preferably operates a software program to process and analyze the data which is collected, in order to compute medical information. The extracted medical information, optionally also with the raw data, is then preferably transferred to the previously described gateway device. The gateway device then preferably relays such information to a remote server, which more preferably is able to provide such information to medical personnel, for example as part of a call center. Therefore, continuous monitoring of the physiological parameters of the user may optionally and more preferably be made, enabling better medical care for the user.

A general, non-limiting example of suitable methods for measuring the heart rate and/or other heart-related physiological parameters of a subject who is wearing the device according to the present invention may be found in the article “Cuff-less Continuous Monitoring of Beat-To-Beat Blood Pressure Using Sensor Fusion”, by Boo-Ho Yang, Yi Zhang and H. Harry Asada—IEEE (also available through http://web.mit.edu/zyi/www/pdf/IEEETrans2000.pdf as of Dec. 9, 2001), hereby incorporated by reference as if fully set forth herein, where systolic and diastolic blood pressure are calculated using the pulse pressure shape per heartbeat. The disclosure does not describe a device which has the functionality according to the present invention, but the disclosed method is generally useful for determining blood pressure from an external measurement of pressure from the pulse through the skin of the subject.

According to exemplary embodiments of the present invention, there is provided a device for measuring at least one physiological parameter of a subject in addition to blood pressure. The device preferably comprises a band for being fastened to a wrist of the user, which is also associated with a blood pressure cuff. The device also features at least one additional sensor for measuring at least one additional physiological parameter of the user, as well as a processor for receiving a signal from the sensor and a signal from the blood pressure cuff and for converting said signals to form medical information. Therefore, the device preferably combines the functionality of a portable blood pressure cuff with at least one other type of physiological measurement in order to assess the medical condition of the user.

Since the device is portable, it may optionally be used for a number of different embodiments. For example, it may optionally and preferably be used for performing a Holter monitoring process, in which one or more physiological measurements (preferably including at least one cardiac physiological parameter) are measured over an extended period of time, such as 24 hours for example. Currently available Holter devices are clumsy and difficult to use, and also may impede daily living tasks of the user. In addition, the present invention also preferably features electronic input and/or display, which enables actions of the user (for example during the Holter task) to be input by the user at least semi-automatically, for example through selection from a menu shown on the display. The display may also optionally (additionally or alternatively) feature such information as reminders about appointments with medical personnel and/or times to take medication, and/or alerts to the user (for example from a medical center).

Preferably, as described above, the device is also in communication with a medical center, to pass the medical information collected about the user to the medical center and also more preferably to transmit information from the medical center to the user through the device.

For implementing the Holter task and/or other types of cardiac measurements, preferably the at least one additional sensor of the device includes an electro cardiogram (ECG) sensor, which preferably at least one electrode associated with the band of the device. Optionally, the band features at least a rigid portion and the electrode is associated with the rigid portion. Also optionally and preferably, the at least one electrode is organized into a set of two or more electrodes, such that only one electrode is required to be in contact with the wrist of the user at any particular time.

The device may also optionally and preferably feature a locator unit for locating the device (and hence the user), in the case that the user is unable to communicate his or her location. The locator unit may optionally comprise a GPS unit.

The principles and operation of a device and method according to the present invention may be better understood with reference to the drawings and the accompanying description.

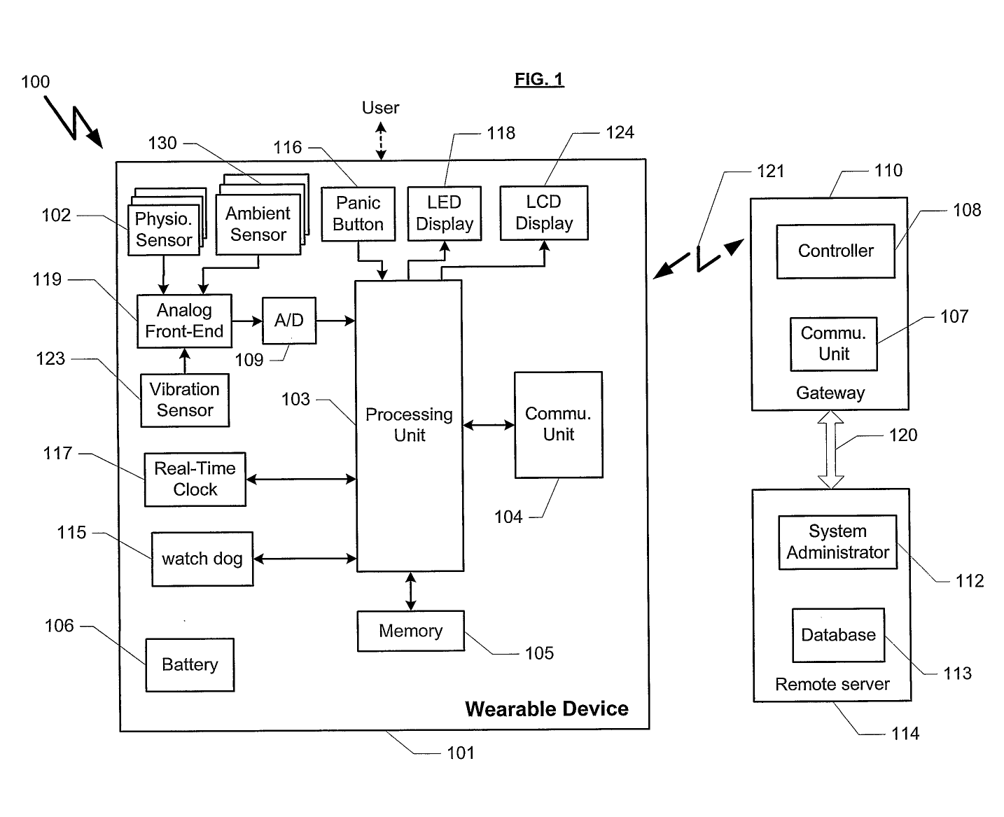

Referring now to the drawings, FIG. 1 is a schematic block diagram of a system according to the present invention. As shown, a system 100 features a wearable device 101 to be worn by a user, preferably as a wrist-mounted device, for example by being attached with a wristband or other fastening article to the wrist of the user. Device 101 features at least one physiological sensor 102 for measuring at least one physiological parameter of the user. The function of an exemplary sensor 102 is described in greater detail below.

The device 101 may optionally feature a vibration sensor 123, preferably a piezoceramic sensor, which is not in direct contact with the skin of the user. Sensor 123 measures the movement of the wrist. The output of sensor 123 can be used by a processing unit 103 to capture the movement of the wrist and to recover some noise received by sensor 102, which is caused by such movement.

Optionally, the output of vibration sensor 123 may record some breathing movements thus enabling the processing unit 103 to calculate the breathing rate, when the user's wrist with the device 101 is placed over the user's abdomen or chest for a period of time.

Device 101 may include additional ambient sensors 130 or additional measuring routines for measuring other parameters. For example, device 101 may optionally have a humidity sensor for measuring the ambient humidity. An exemplary humidity sensor may be the Humidity Gauge manufactured by Honeywell.

In order to support processing of the measured physiological parameter or parameters, processing unit 103 may optionally include internal RAM and non-volatile program memory (not shown). Also processing unit 103 may optionally include an extended data memory 105 located externally to processing unit 103. Processing unit 103 preferably executes at least one instruction for processing the data obtained by sensor 102.

Examples of such processing units 103 include but are not limited to MSP430 by TI company, which contains some channels of 12 bit A/D converters, a 2K bytes of internal RAM and 64K Bytes of non-volatile program memory.

Extended memory component 105 is preferably an electrically erasable non-volatile serial external memory component. Examples of such a memory component include but are not limited to FM24CL64-S (Ramtron, USA), with 64 Kbit of fast access read/write serial memory for storing temporary data related to the sampled physiological parameter.

Device 101 may optionally feature a real time clock 117 in order to provide an accurate time and date for each measurement, as device 101 can optionally store a few measurements before transmitting such data and/or information to a gateway device 110, as described in greater detail below. Stored data and/or information may also optionally be used for such applications as reminding the subject to take medication, perform a prescheduled measurement, and so forth. An A/D converter 109 with multiple inputs is also optionally and preferably present if sensor 102 is an analog sensor, in order to convert the analog signal to a digital signal.

Device 101 preferably features an internal communication unit 104, for at least unidirectional, but more preferably bi-directional, communication with gateway device 110. Gateway device 110 may feature a communication unit 107. Communication unit 104 may optionally communicate with communication unit 107 through a wire or alternatively through a wireless communication link 121. According to a non-limiting exemplary embodiment of the present invention, gateway device 110 is located relatively close to the user and hence to device 101, for example by being located at the user's premises. As a non-limiting example, gateway device 110 could optionally be installed in the home of the user.

Gateway device 110 also optionally and preferably features a controller 108 for controlling functions of gateway device 110, such as communication with device 101 for example.

Gateway device 110 preferably communicates with a remote server 114 through a data link 120, which could optionally be a direct dial-up modem connection with DTMF coding or TCP/IP using regular LAN or modem connection to an ISP, for example. In any case, data link 120 may optionally be a wired or wireless link, for example through a cellular telephone and/or land-based telephone system, or a combination thereof.

Remote server 114 may be controlled by a system administrator 112, which may be a person (for manual operation) or a software program (for automatic operation), or a combination thereof. Remote server 114 also preferably features a database 113 for storing data received from gateway device 110.

Device 101 may also feature a manually operated panic alarm button 116 to be manually activated by the user, for example if the user is in distress. Device 101 may also optionally feature a LED display 118, for example in order to indicate of alert activation or a low battery level.

Physiological sensor 102 is preferably part of a sensor assembly. Without the intention to limit in any way, the following discussion centers on such a physiological sensor 102, which contains a piezoceramic transducer for generating an electrical signal, having amplitude corresponding to the magnitude of applied pressure. Therefore, if at least a portion of the transducer is located adjacent to, or fasten by a fastening article to, and in physical contact with, an area of the wrist where blood pressure pulses may be detected, the transducer generates electrical pressure pulses corresponding to the detected blood pressure pulses. Each of the electrical pressure pulses preferably defines a maximum voltage over a systolic interval and a minimum voltage over a diastolic interval.

Although a piezoceramic sensor is used as a force transducer according to a preferred embodiment of the invention, it should be appreciated that other transducers known to the art may be employed without departing from the spirit of the invention. Examples of such sensors include but are not limited to piezoelectric transducers, resistive strain gauges and pressure sensor.

The piezoceramic transducer is desirable for the present invention since the transducer measures the direct effect of the pressure exerted within the radial artery, while other transducers, for example resistive strain gauges, measure secondary effects such as the strain forces that are applied at the surface of the skin due to the expansion of the radial artery. Piezoceramic transducers are also cheaper than piezoelectric transducers but still produce a high-quality signal.

As shown with regard to FIG. 1, the analog output of sensor 102 is first preferably treated by an analog front-end 119, which more preferably contains analog selector to select the appropriate sensor followed by an analog filter (not shown). As a non-limiting example, this analog filter preferably has a cutoff of about 20 Hz, a linear phase response, a flat amplitude response up to 10 Hz and an amplification of about 3 for acquiring the full spectrum of a typical blood pressure pulse. The filtered signal then enters A/D converter 109.

Processing unit 103 preferably controls the operation o f A/D converter 109. When a physiological measurement is initiated, A/D converter 109 starts sampling the filtered analog signal of sensor 102 from analog front-end 119, preferably at a rate controlled by processing unit 103. This rate is optionally and more preferably 80 samples per second as to over sample the data by a factor of 4 to maintain a good quality sampled signal. A/D converter 109 preferably transfers the analog data into a digital coded word, optionally at resolution of 12 bits per sample, for example.

An exemplary measuring period may be about 50 seconds in which data is gathered at processing unit 103. Processing unit 103 preferably operates a software program for examining the validity of the sampled data, in order to determine whether the data contains some indications of legitimate physiological data (such as of a blood pressure pulse of an artery) or alternatively whether the data contains only noise or poor readings. In the second case, A/D converter 109 preferably starts sampling the signal again in order to obtain data for measurement. This process preferably continues until the software determines that sufficient valid data has been collected or after a few successive rejections (usually after 3 times).

Then, the software program preferably performs an algorithm for calculating some medical parameters from the sampled data, such as the calculation of systolic and diastolic blood pressure using a method as disclosed in U.S. Pat. No. 4,418,700, which is hereby incorporated by reference as if fully set forth herein. The software program may be also located within gateway and/or the remote server.

The calculated parameters are then preferably stored in memory 105. The data stored in memory 105 is preferably transmitted to gateway device 110 periodically, or alternatively or additionally after manual operation of panic button 116.

The calculated parameters are also optionally and preferably displayed on a local display 124 such as LCD, so the user can view the last medical results locally.

More preferably, data for all medical parameters that are sent to remote server 114 are sent according to a security protocol (such as but not limited to HIPAA protocol) for maintaining the privacy of the user.

Furthermore, the software program preferably performs another algorithm for generating an alert if the medical parameters have values beyond or otherwise outside of the normal expected values.

Although a one-way link from device 101 to gateway device 110 may be used, device 101 preferably features a two-way communication link as shown for link 121, for establishing more reliable communication with gateway device 110. Examples of communication units 104, 107 include but are not limited to an RF401 UHF transceiver (Nordic), which operates in the universal ISM band (433.92 Mhz), an infrared transceiver, and a “Bluetooth” protocol enabled-transceiver operating bi-directionally in the 2.4 GHz band.

Device 101 preferably has its own unique identifier, stored in non-volatile data storage, more preferably in memory 105. Each time device 101 sends a wireless message to gateway device 110, device 101 also preferably sends the unique identifier to gateway device 110, although optionally the identifier may be sent only periodically, for example once per day. Gateway device 110 also preferably sends a message to a particular device 101 by including the device identifier in the message, thereby specifying which such device should receive the message.

As previously described, device 101 preferably has its own real time clock 117. For periodic monitoring of the user, real time clock 117 is preferably used to provide a time tag for each set of results. This time tag is very important for continuous monitoring of the user for long periods of time. By examining the data recorded over of the user for long period of time, a change or alteration in the health condition of the user may be detected. Real time clock 117 may optionally be implemented by separate hardware such as RTC8564 (EPSON, US) for example, or alternatively by a software program for operation by processing unit 103.

In some embodiments of device 101 the output of real time clock 117 may be displayed on one of displays 118 or 124 for displaying the date and time.

Device 101 may also optionally feature a watchdog 115, which monitors the function of device 101. If the end of a watchdog time period is reached, device 101 is assumed to have a fault in its operation, and a master reset is preferably initiated automatically.

Device 101 also preferably features a power source such as a battery 106, which powers device 101. Examples of suitable batteries include but are not limited to the silver oxide coin battery model 386 (Panasonic, Japan) having 150 mAh in capacity with a pulse burst of 75 mA for a short period of time (about 5 sec for each pulse). Battery 106 optionally and preferably contains enough energy to power the device for more than one year of operation without being replaced.

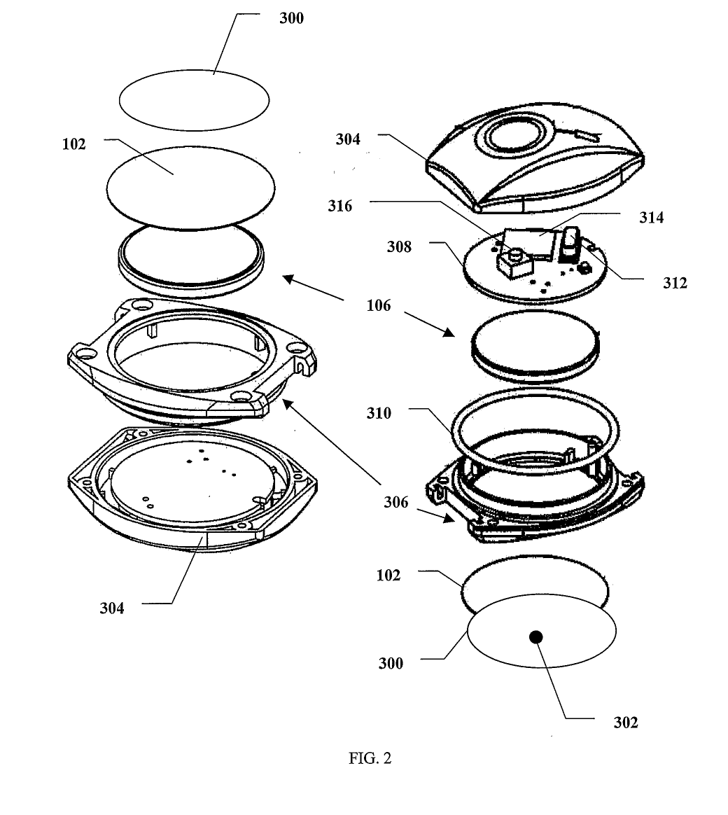

FIG. 2 shows an exploded view of an exemplary device according to FIG. 1. As shown, the device features sensor 102, shown with the preferred but exemplary implementation of a piezoceramic sensor as previously described. The device also optionally and preferably features battery 106, and a push button 316 (for optional implementation of the panic button of the device of FIG. 1). Battery 106 may optionally be replaced with a plurality of smaller batteries (not shown). The device preferably features a processor 314 (which may optionally be similar or identical to the processing unit of the device of FIG. 1. The components of the device are preferably held by a case 306.

For this exemplary implementation, sensor 102 is in physical contact with an anvil 300 via a protrusion 302. Protrusion 302 is welded, optionally by a laser, on one side to the center of anvil 300 and on the other side to the center of sensor 102. Anvil 300 is pressed against the skin of the wrist of the subject (not shown), more preferably at a pulse point. Anvil 300 may optionally be a rigid disk made for example of polymer, or optionally a metal, such as gold plated copper or stainless steel, for example. Of course, any other type of suitable material, or combinations of materials, may also optionally be used. Anvil 300 therefore collects and integrates the pressure waves, which are associated with each pulse of the blood of the subject, from the area below anvil 300. This pressure is preferably transferred from the center of anvil 300 to the center of sensor 102 via protrusion 302. Sensor 102 then emits voltage to form a signal, preferably according to a linear output. By using this architecture, the present invention may measure the blood pressure pulse without blocking the blood flow in the artery.

This signal is then received by processor 314, which preferably extracts medical information from the measurement of the physiological parameter. Processor 314 optionally and preferably features a crystal oscillator 312, for stabilizing the internal clock of processor 314. Processor 314 may communicate with the real time clock of the device (not shown). Also not shown are the extended memory, transceiver (communication unit), A/D converter and analog front end of the device.

Processor 314, oscillator 312 and push button 316 are all preferably mounted on a PCB board 308. PCB board 308 is then preferably sandwiched between battery 106 and a device cover 304. Device cover 304 preferably features a soft portion, which may be rubber for example, for enabling the user to locate and depress the panic push button through push button 316.

An o-ring 310 is preferably used for waterproof sealing between cover 304 and the case 306 of the device. Anvil 300 then is held between sensor 102 and the skin of the user (not shown), for example.

According to an alternative implementation of the device of FIGS. 1 and 2, sensor 102 and anvil 300 could optionally be located in the wristband for affixing the device to the wrist of the user (not shown).

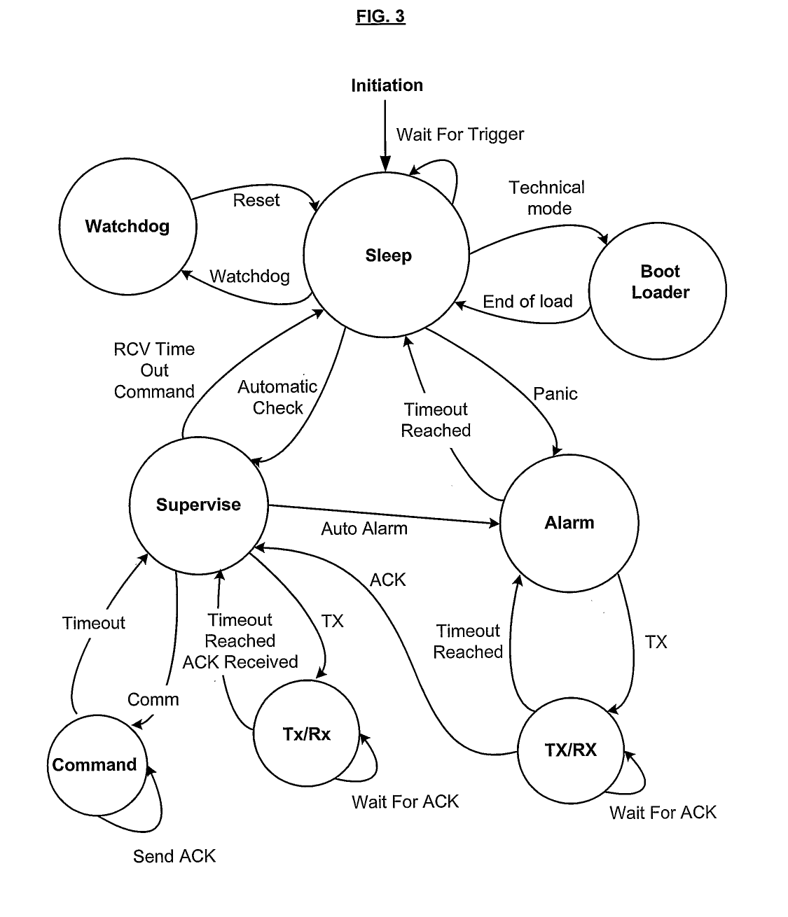

FIG. 3 is a state flow chart of the operation of the device. As the device software begins operation for the first time, the software preferably makes some initializations using default values. Once the device has been initialized, the software preferably triggers a watchdog function shown as a “Watchdog” process, and then enters a sleeping mode for saving battery life, shown as a “Sleep” process.

If the end of a watchdog time period is reached, the device is assumed to have a fault in its operation, and a master reset is preferably initiated automatically. The device is preferably “woken up” according to one of three triggers. First, the device is preferably woken up when the user presses an activation button (the panic button 116 in FIG. 1 can be used as the activation button) manually for a few seconds. This process is shown by the “Alarm” state. The device then preferably immediately starts a transmission to the gateway device, containing a distress indication and the device identifier. Then the device enters a receiving mode for a few seconds, waiting for acknowledge (ACK) from the gateway device. This process is shown as a “TX/RX” state.

If the acknowledge message is not received within this period of time a repeated message is initiated. Additional transmissions are initiated, if necessary. However, if after a predefined number of repeated times an acknowledge message is not received, an error message is stored within a log and no more tries are made. More preferably an indication is displayed on the display screen for a few seconds, optionally with an audible alarm. Then, the process returns to the “Sleep” state. However, if the received ACK contains no commands the device returns to the “Sleep” state, otherwise the device does the command and sends an ACK to the gateway. The gateway returns an ACK with another command to continue or without a command to terminate this process. After doing the last command the device returns to the “Sleep” state.

Second, when the user presses the activation button manually for a short period of time, the process turns to “Supervise” state, where the device collects data from its sensors, preferably calculates some medical information concerning the current physiological status of the user. Then, the device turns into “Tx/Rx” state, where the device transmits a message containing the identifier, and the raw measured data and/or the calculated medical parameters. And if the received ACK contains no commands the device returns to the “Sleep” state, otherwise the device does the command and sends an ACK to the gateway. The gateway returns an ACK with another command to continue or without a command to terminate this process. After doing the last command the device returns to the “Sleep” state.

In the third case where the device exits its “Sleep” state, an external real time clock signals the device to execute an automatic check. Then, the process enters “Supervise” state as discussed in the above paragraph, only that this time for saving battery life, the device initiate the “Tx/Rx” process only once for a few successive times sending all the accumulated data in one transmission. Then, the device preferably enters a “Sleep” state unless the measured parameters exceed a predefined threshold at least once, but preferably for a few successive measurements. In this case, the device initiates an automatic alarm entering the “Alarm” state, if the device has permission to do so, as previously described.

When a timer for a supervise process has been running or after an alarm, the device preferably exercises an automatic check as described above, and after that initiates a transmission to the gateway device including all the data collected after the last transmission. Then the device preferably waits for acknowledge, preferably repeating the transmission again if not receiving such an acknowledge message. In the acknowledge message, a command for the device can be stored. In such a case the device performs this command and then the device sends an acknowledge message to the gateway device. This process may optionally continue until an acknowledge message without a command is received, after which the device preferably returns to sleep mode.

Other exemplary embodiment may use additional routines and modes, such as a mode that verifies whether the user is in the user's premises for example. This mode is optionally initiated every few minutes and transmits a short transmission to the gateway. The gateway waits for those signals and if in a certain window of time, for example 30 minutes, a transmission has not been received, the gateway calls the medical center and reports that the user is missing.

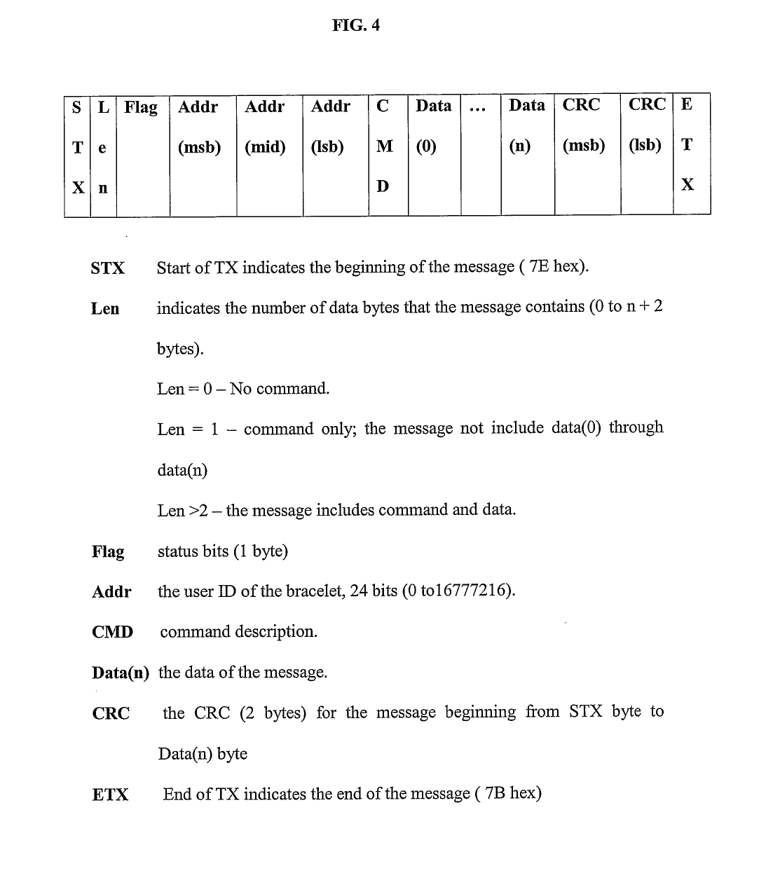

FIG. 4 describes an exemplary message format for exchanging messages between the device and the gateway device. Every message preferably starts with a preamble STX byte (hex 7E), followed by a byte which contains the number of bytes in the current message, and three bytes of address, followed by a command byte and its corresponding data bytes. This is followed by two bytes of CRC and an ETX byte (hex 7B).

As such, the message is a variable length message with strong error detection and correction method for enhanced communication reliability. Each message optionally and preferably contains a low battery indication, if necessary.

In case of a unidirectional communication link between the device and the gateway, a repeated message is preferably transmitted for a predefined number of times, such as 20 times for example, after which the device preferably enters a sleeping mode if no answer is received.

In case of a bi-directional link, for each message sent to the gateway device, an acknowledge message is preferably returned by the gateway device and vise versa. This message may also contain a command for the device encoded in the CMD byte within the message. Commands could optionally include, but are not limited to, one or more of the following:

1) Get/Set service type

2) Get/Set device ID

3) Set interval between successive medical checking

4) Set interval between successive supervision transmissions

5) Set Time and date

6) Set threshold for automatic alerts

7) Set device calibration

Each time the device sends a message to the gateway, the device may optionally contain a Battery OK/Battery Low indication for the battery situation. This signal preferably appears three months before the battery finishes, enough time to ask the user to replace the battery.

Each time the device sends a supervise-type message to the gateway, the device preferably sends also all the medical data stored in its memory with that message.

Each time the gateway device sends a command back to the device, the device preferably returns an acknowledge message with a 3 bit message serial number to the gateway device, in order to fulfill a full handshake between the two. If the gateway device does not receive acknowledge from the device within a few seconds, the gateway device preferably sends its transmission message again with the same serial number. The message may even be repeated a few times, each time waiting for acknowledge. If acknowledge is not received, a logbook is updated with an error message, and more preferably an indication LED is turned on for error indication.

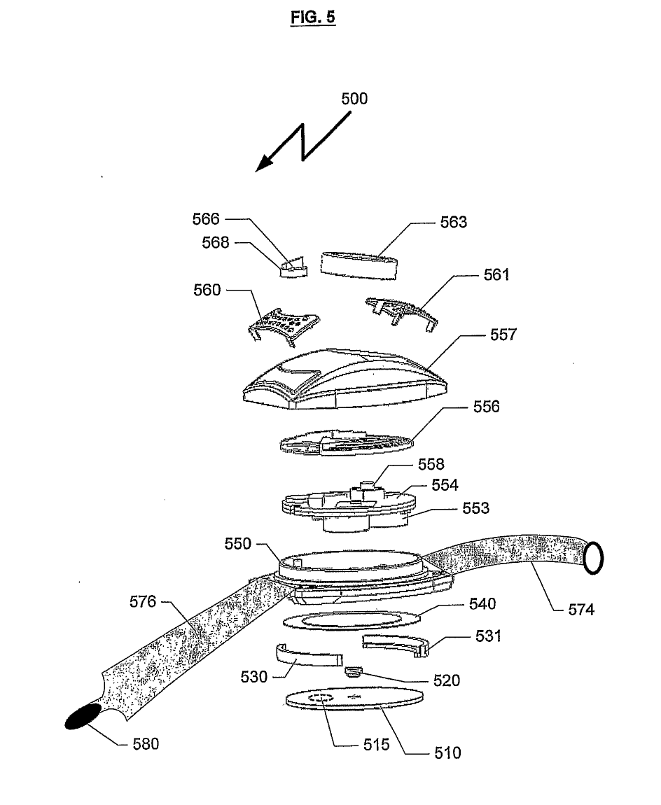

FIG. 5 shows an exploded view of a device 500 according to exemplary embodiments of the present invention. In addition to, or in place of, measuring blood pressure, device 500 may optionally measure other activities of the body including but not limited to ECG, tonus activity, breathing rate, body temperature and the SpO2 (oxygen saturation in the blood) value in the blood of the user, for example.

Device 500 in FIG. 5 may be similar to an expanded wristwatch in shape, where bottom anvil 510 is the section which lies flat against the wrist. This forms the base of device 500 whose center is lower case 550. All other components are built onto lower case 550, culminating at the top with face-plate 557, upon which are mounted a number of additional components including sensors.

Sensor 540 is optionally and preferably attached to lower case 550 of device 500 by two arcs 530 and 531. Each arc 530 and 531 preferably has a vertical portion and a horizontal portion. The horizontal portion is preferably placed between sensor 540 and anvil 510, and is pressed against lower case 550 holding sensor 540 in place. The vertical portions of arcs 530 and 531 are preferably affixed into an appropriate slot in lower case 550 of device 500.

Lower case 550 may optionally have one or more electrical boards 554 and 556 that comprise the electrical circuitry, which is disclosed in conjunction to in FIG. 1 and or FIG. 5, of the device including batteries 553. A vibration sensor (an accelerometer) may optionally be connected to one of boards 554 or 556.

Device 500 is preferably covered by a top cover 557, that optionally and more preferably has two electrodes 560 and 561, SpO2 sensor 566 and optionally a single push panic button 558 that is preferably pressed by the user upon commencement of a measurement period, or if the wearer presses panic button 558.

Pressing the flexible portion 563 within top cover 557 causes panic button 558 to be pushed, and preferably initiates an automatic process within device 500, then device 500 may initiate the panic thread and/or optionally start a measuring thread and transmits a set of results to gateway 110. Gateway 110 optionally and preferably stores those results and upon establishing the connection with the call center, gateway 110 transmits the results to the call center. The panic thread starts by establishing a connection with the call center via gateway 110 (FIG. 1).

In other embodiments, the panic thread starts upon pressing activation push button 558 for long period of time (e.g. above few seconds, 5, 6 etc.), thereby initiating a call to medical center. In contrast, pressing activation push button 558 for a short period of time, for example shorter than a second, starts an automatic measuring thread. It should be noted that the terms “activation push button”, “panic push button”, “panic button” or “push button” may be used interchangeably herein The measuring thread optionally and preferably starts by scanning the available sensors 102 for a first sensor 102 that produces a valid signal (see FIG. 1). A valid signal is defined as a signal that meets predefined requirements including but not limited to, one or more of the signal amplitude being within a certain range, frequency being within a certain range and so forth. The valid signal is processed by the appropriate analog front-end 119 and processing unit 103 (see FIG. 1).

Upon receiving the awakening signal from a timer within the real time clock, device 500 may inform the user that a measuring process is initiated. Upon terminating the measurements, the results are sent to the remote server 114 (FIG. 1) via gateway 110.

Two bands 574 and 576 are optionally connected to lower case 550 and are preferably used to fasten the device to the wrist of the user. The long band 576 may optionally have a flexible conductive wire (not shown) which functions as an antenna, and which is connected to the transmitter of the communication unit 104, inside device 500, while the far end of long band 576 may comprise temperature sensor 580 connected by pair of wires (not shown) to the internal circuitry, both of which are described in greater detail below.

Device 500 may optionally be used to measure blood pressure pulse using piezoceramic transducer 540 to generate an electrical signal. The amplitude of the electrical signal from piezoceramic transducer 540 corresponds to the magnitude of pressure applied thereto. Piezoceramic transducer 540 may be a common piezoceramic buzzer, made of PZT material, and may optionally and additionally be used as a common buzzer, which receives the alarm signals from processing unit 103 (see FIG. 1) and produces the alarm sound. The alarm sound is generated by forcing voltage over piezoceramic transducer 540, which then buzzes for the duration of the alarm signal.

The exemplary sensor for sensing blood pressure pulse preferably comprises three elements: anvil 510, protrusion 520 and piezoceramic transducer 540. Protrusion 520 is preferably welded, optionally by a laser, on one side to the center of anvil 510 and on the other side to the center of piezoceramic transducer 540. Anvil 510 is pressed against the skin of the wrist of the subject (not shown), more preferably at a pulse point. Anvil 510 may optionally be a rigid disk or other structure, made for example of polymer, or optionally a metal, such as gold plated copper or stainless steel, for example. Of course, any other type of suitable material, or combinations of materials, may also optionally be used.

Anvil 510 therefore collects and integrates the pressure waves, which are associated with each pulse of the blood of the subject, from the area of skin below anvil 510. This pressure is preferably transferred from the center of anvil 510 to the center of piezoceramic transducer 540 via protrusion 520. Piezoceramic transducer 540 then emits voltage to form a signal, preferably according to a linear output. Protrusion 520 preferably is able to focus the input pressure, therefore increasing the output signal of piezoceramic transducer 540.

Therefore, if at least a portion of anvil 510 is located adjacent to, and in physical contact with, an area of the wrist where blood pressure pulses may be detected, transducer 540 generates electrical pulses corresponding to the detected blood pressure pulses. Each of the electrical pressure pulses preferably defines a maximum voltage over a systolic interval and a minimum voltage over a diastolic interval. The electrical signal from transducer 540 is preferably amplified by analog front end 119 and transferred via A/D converter 109 to processing unit 103 (see FIG. 1). Processing unit 103 processes the digital signal and may deliver a plurality of medical information based on the measurement of blood pressure pulse including but not limited to heart rate, regularity in heart rate, breathing rate, arrhythmia of the heart (if any), general rhythm and functioning of the heart as well as the blood pressure amongst others.

Device 500 optionally and preferably features two conductive areas 560 and 561 at the top. In the bottom part of device 500, anvil 510 preferably has a conductive area 515, which preferably sits adjacent to the skin of the user. In some exemplary embodiments, conductive area 515 may cover the whole of anvil 510, a non-limiting example of which is constructing anvil 510 of metal. Each of conductive areas 560, 561 and 515 is preferably electronically connected, as one of the sensors 102, to an analog front-end 119 (FIG. 1).

Conductive areas 560, 561 and 515 may optionally and preferably be made of metal, polymer coated with a conductive layer or any other conductive material including but not limited to gold plated copper. Conductive areas 560, 561 and 515 form three electrodes that may be used for measuring electrochemical activity of the user's body (e.g. ECG, or tonus activity). This activity measures the effects of electricity on chemical and biological activities in the body, and is referred to hereinafter as electrochemical activity.

For optionally measuring ECG, the user has to touch, simultaneously, the two conductive areas 560 and 5 61 with the user's second hand, for example with two fingers, to form three measuring points including the skin portion, on the first hand, that is adjacent to conductive area 515. The three electronic signals from conductive areas 560, 561 and 515, are transferred to analog front-end 119 (FIG. 1). Analog front-end 119 extracts the ECG analog signal from the three signals by using the signal of one electrode as a reference and amplifying the differential voltage between the other two electrodes. The ECG analog signal is then transferred to A/D converter 109 and from there the digital ECG signal is transferred to processing unit 103 (FIG. 1). Analyzing the analog signal to extract the ECG signal may be done by electrical circuits that are known in the art.

Additional medical information may be determined from the ECG signal. For example, information about breathing rate may be processed based on methods that are described in the prior art. An exemplary method is disclosed in the following article: “Derivation of Respiration Signals from Multi lead ECGs”. By George B. Moody, Roger G. Mark, Andrea Zoccola and Sara Mantero. This article originally appeared in Computers in Cardiology 1985, vol. 12 pp. 113-116 (Washington, D.C.: IEEE Computer Society Press), which is hereby incorporated by reference as if fully set forth herein.

Other medical information that may be produced by processor unit 103 (FIG. 1) is the Pulse Wave Transit Time (PWTT), that may be determined by measuring the time delay between the electrical pulse of the heart, measured from the ECG signal and the time of the blood pressure pulse.

In another exemplary embodiment, A/D converter 109 (see FIG. 1) may be integrated into processing unit 103. Processing unit 103 processes the ECG signal and generates medical information such as, but not limited to, heart rate, regularity in heart rate, breathing rate, arrhythmia of the heart (if any), as well as the general rhythm and functioning of the heart for example. The medical information is then transferred to the call center via gateway 110 (FIG. 1).

Device 500 may optionally be used for measuring the oxygen saturation in the blood (SpO2) by using SpO2 sensor 566. Sensor 566 optionally and preferably has two light sources, optionally by two LEDs (light Emitting Diode) and a photoelectric detector for example. One of the LEDs emits in the infrared band and the other emits in the red band.

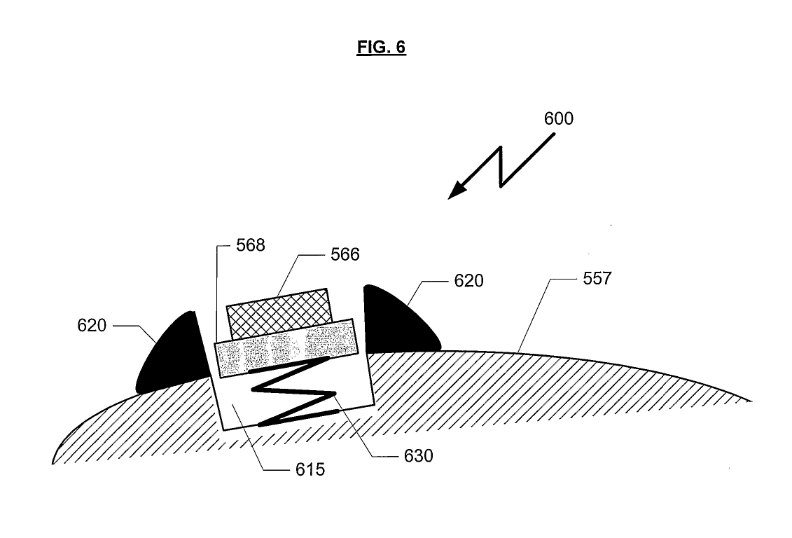

FIG. 6 is a system diagram of an exemplary method of placement of SpO2 sensor 566 in faceplate 557 (of device 600). The two LEDs and the photoelectric detector (not shown here) of SpO2 sensor 566 are optionally installed over platform 568 which is supported by flexible support 630. Support 630 may optionally be any material which can absorb and exert pressure, including but not limited to a spring, piece of rubber, a sponge, flexible wing and so forth. Support 630 is locked in a niche 615 in faceplate 557. The edge of niche 615 is optionally and preferably surrounded by material 620, which is more preferably flexible and opaque. Material 620 may optionally be any flexible opaque substance including but not limited to rubber, sponge, flexible wings and so forth.

To perform SpO2 measurement, the user presses a finger against sensor 566, thereby pushing sensor 566 and platform 568 against flexible support 630 in the direction of faceplate 557. Flexible support 630 absorbs part of the force by moving inside niche 615 and responding to the pressure with a predetermined force, which is a result of the mechanical properties of flexible support 630. The force is predetermined to as to avoid disturbing the blood flow in the tissue. The skin of the finger (not shown) that surrounds sensor 566 is therefore pressed against flexible opaque material 620, thereby b locking light creating a d ark space around the measuring area which prevents the surrounding light disturbing the measurement process. Upon depression of sensor 566, processing unit 103 (FIG. 1) initiates the SpO2 measuring thread. Processing unit 103 instructs the current drivers in analog front-end 119 (FIG. 1), which is associated with sensor 566, to force current through the LEDs alternately in sensor 566. The reflected light from the finger is received by the photo detector, which converts the photons into electronic signal. The electronic signal is fed, as one of sensors 102, to analog front end 119. Analog front-end 119 processes the analog signal and transfers the processed analog signal to A/D converter 109 (FIG. 1). The digital signal is transferred to processing unit 103 (FIG. 1), which processes the digital signal and generates the SpO2 figure. This information is then transferred to the call center via gateway 110 (FIG. 1).

The signal that is collected from the SpO2 sensor may also optionally be used for producing other heart related information. For example, processing the signal that reflects the intensity of the reflected IR light may produce information such as heart rate, PWTT, irregularity of heart rate etc.

Other exemplary embodiments may have the SpO2 sensor installed instead of the blood pressure pulse sensor (anvil 510, protrusion 520 and piezoceramic sensor 540). In this embodiment the reflected light is received from the wrist instead of the finger.

Returning to FIG. 5, device 500 may optionally have a temperature sensor 580 which is installed at the long band 576. Temperature sensor 580 preferably includes a thermistor located in a metal cup and is connected via two flexible conductive wires (not shown) that run along the band into the lower case of device 500. The two wires are connected as one of sensors 102 (FIG. 1) to analog front-end 119. Analog front-end 119 converts the changes in the resistance of the thermistor into an electrical signal with magnitude proportional to the temperature of the user. The analog signal is converted into digital signal by A/D Converter 109 and transferred to processing unit 103. Processing unit 103 converts the digital signal into temperature information and sends this temperature information via gateway 110 to the call center.

Temperature sensor 580 is preferably installed in a protected solid housing. The solid housing may optionally be made of polymer, metal, gum or any material able to provide the necessary properties.

To start measuring the temperature of the user device 500 is optionally removed from the user's hand and sensor 580 is preferably pressed against the user's armpit (not shown).

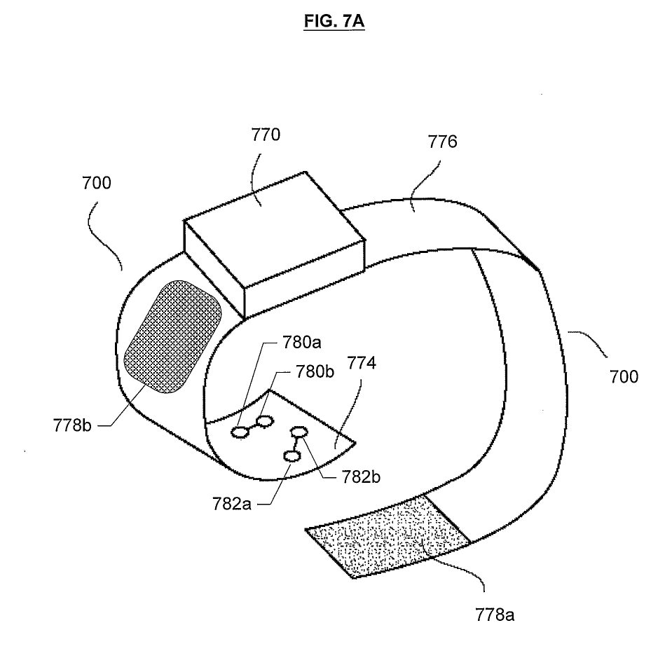

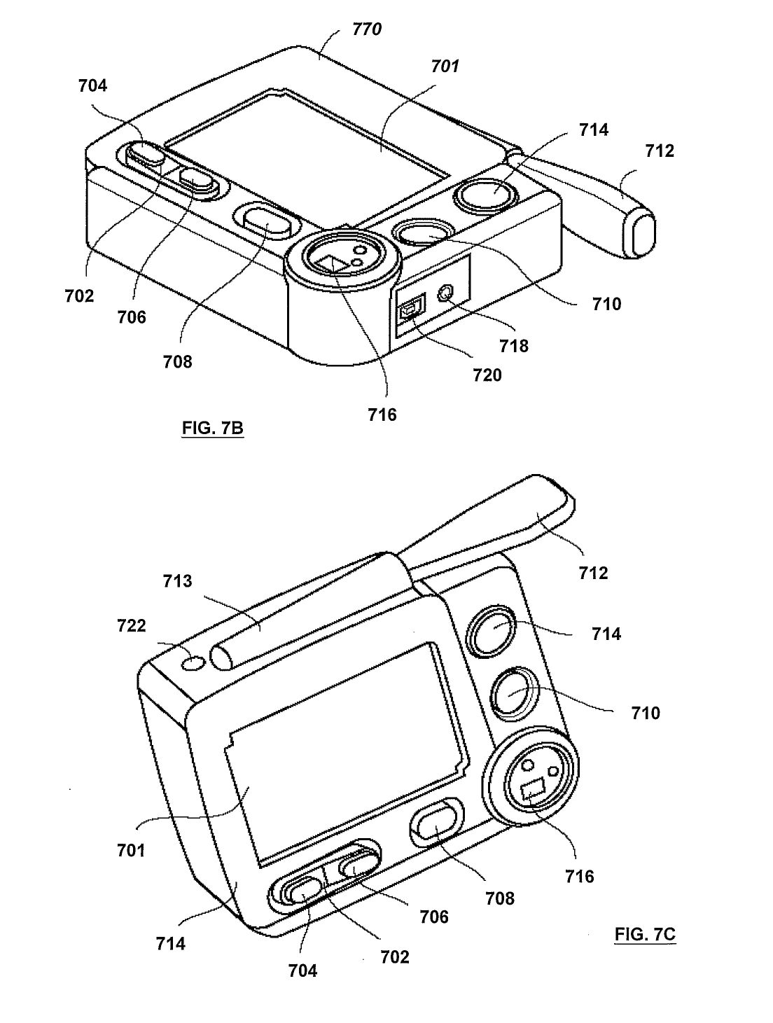

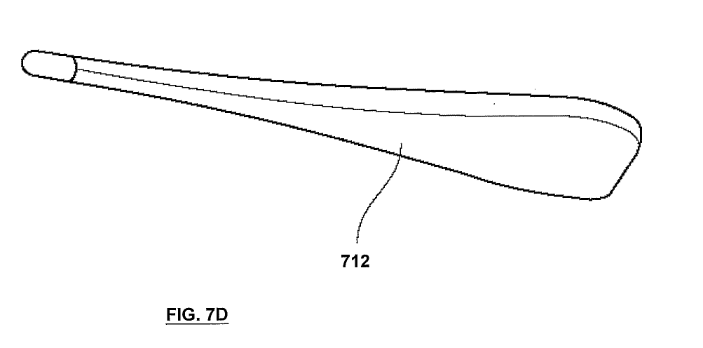

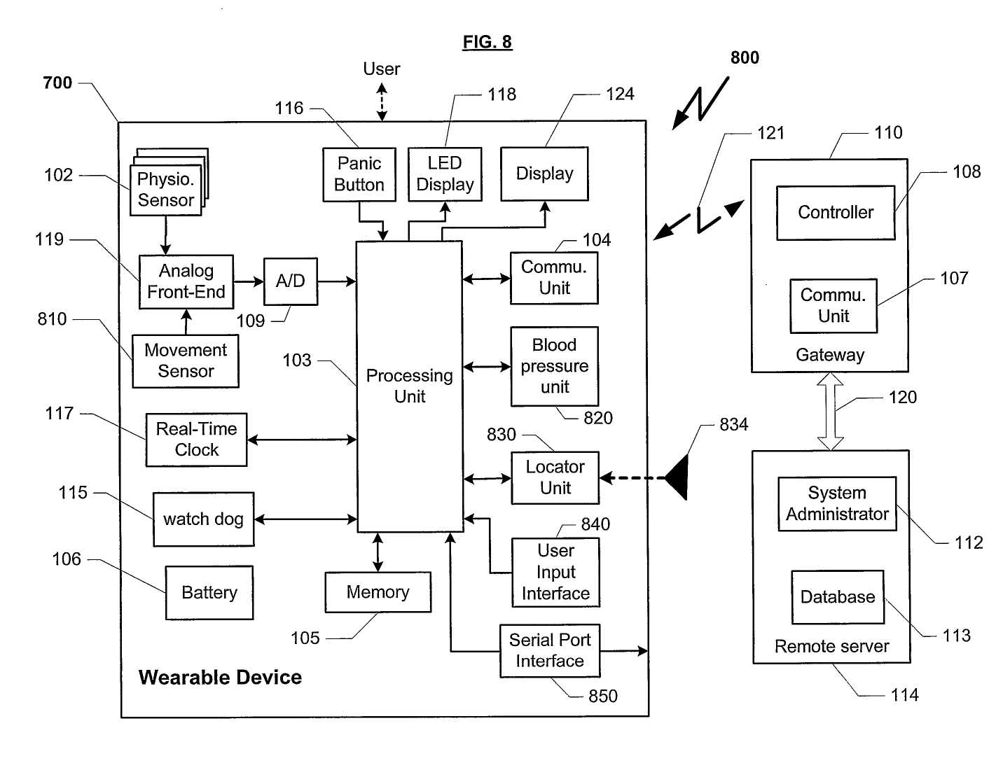

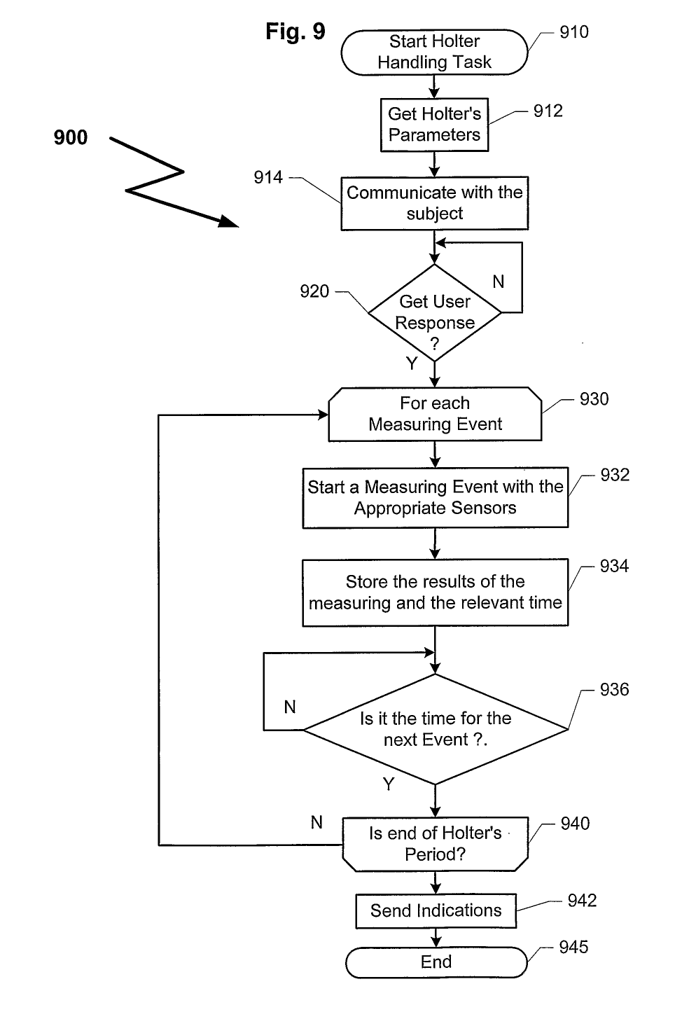

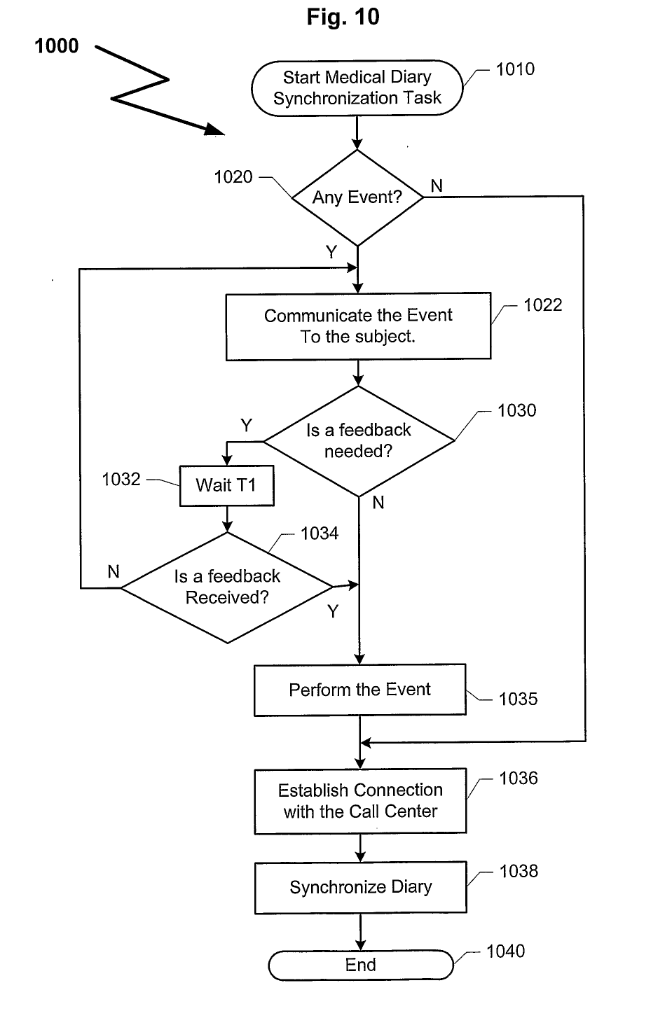

According to other embodiments of the present invention, the device according to the present invention may optionally be combined with a blood pressure monitoring device, such as a Wrist Blood Pressure Monitor that use Oscillometric Measurement Method with a cuff, for example. FIGS. 7-8 show different aspects of such an exemplary device according to the present invention. FIG. 9 shows a flowchart of an exemplary extended physiological monitoring task (a Holter task) which is preferably performed with the device of FIGS. 7-8, but which optionally may be performed with the device according to the present invention as described in accordance to other embodiments above. FIG. 10 shows a flowchart of an exemplary method according to the present invention for synchronizing a medical care management function of the device according to any of the above embodiments with a central care facility.

Turning now to FIG. 7A, which is a schematic diagram of another illustrative embodiment of a device according to the present invention, the illustrative device is shown with an additional blood pressure monitoring device according to the present invention. FIG. 7A shows a wearable device 700, combined with a blood pressure cuff that is associated with the band of the wearable device 700. The band may be comprised of two sections, a flexible band or section 776 and a rigid band or section 774. The blood pressure cuff may be associated with the flexible band or section 776 or with both bands or sections. Blood pressure cuff preferably features two sets of electrodes, electrodes 780A, B and 782A, B. The electrodes may be located at the rigid (or semi rigid) section 774, that is part of the band as shown. By rigid or semi-rigid, it is meant that the section is sufficiently hard surfaced but flexible enough to be able to conform to the shape of the wrist, while still being able to resume its original shape after removal from the wrist.

Electrodes 780A, B and 782A, B are preferably held against the skin of the subject (not shown) by a fastener 778A, 778B, which more preferably maintains the blood pressure cuff, which is associated with the band 776 and/or 774, as a closed or semi-closed loop around the wrist of the subject (not shown). Fastener 778A, 778B may optionally be a Velcro device for example. The electrodes at each one of the sets (780 or 782) are connected in parallel. For example electrode 780a is connected in parallel to electrode 780b. The present invention is not limited to two electrodes in one set. Other embodiment may use other number of electrodes in one set. Using more than one electrode in a set improves the electrical contact with the skin of the subject within any given moment. A third electrode 714 (FIG. 7B) is preferably located on the MACU 770 top case, to be touched by the user's other hand.

Device 700 also preferably features a monitor and control unit (MACU) 770, which preferably features at least a blood pressure monitoring and control unit. MACU 770 also preferably includes one or more sensor(s) and more preferably one or more analyzer(s) for at least measuring, but more preferably for also being capable of monitoring, at least one other type of physiological parameter including but not limited to ECG (electro cardiogram), SpO₂, breathing rate, etc. The ECG may be monitored through ECG Electrodes 780A, B and 782A, B and 714.

MACU 770 preferably also includes functionality for monitoring blood pressure and/or other physiological parameters over an extended period of time of at least a few minutes (at least about 2 minutes), more preferably at least a few hours (at least about 2 hours) and most preferably at least about 24 hours (which may for example optionally be implemented according to a Holter functionality).

Other examples of sensors, which may optionally be provided with MACU 770, include but are not limited to, body temperature, SpO₂ (oxygen saturation in the blood), as described in greater detail above, and an accelerometer (movement sensor) for measuring breathing. The function of the accelerometer is described in greater detail below.

FIGS. 7B-7D are diagrams of external views of different parts of the illustrative device 700 according to the present invention. FIGS. 7B and 7C show diagrams of two views of MACU 770, while FIG. 7D shows a diagram of a temperature sensor that may optionally be used with device 700.

As shown with regard to FIG. 7B, MACU 770 preferably features a screen 701 for displaying a user interface, which is more preferably a GUI (graphical user interface). The user interface preferably features a plurality of menus with one or more choices, and/or other graphical selection features, thereby enabling the user to select, manipulate or manage one or more functions of device 700. Preferably, the user also enters an identifier before being able to select, manipulate or manage one or more functions of device 700, as described above. Screen 701 also optionally and preferably displays information to the user, for example about one or more physiological parameters being measured or monitored, as well as optionally about one or more medical care functions (such as when a particular medication should be administered for example), as described in greater detail below.

In order to assist the user to operate device 700, one or more buttons are preferably provided for enabling the user to enter a command to MACU 770. As shown for the purposes of illustration only and without any intention of being limiting, these button(s) may include, but are not limited to, one or more of a cursor button 702 (preferably including separate cursor up and down movement buttons 704 and 706 respectively as shown); an enter button 708 for entering a command and/or information; and a panic button 710.

Device 700 also preferably features a temperature sensor 712 for measuring the temperature of the user (shown in greater detail with regard to FIG. 7C), more preferably under the tongue (in the mouth), in the armpit and/or in the rectum of the user. Temperature sensor 712 is optionally and preferably inserted into a holder 713 on MACU 770 for storage (shown in more detail with regard to FIG. 7B) the temperature sensor is connected to the MACU 770 by cable with connector at the end, which is connected to connector 722 (FIG. 7C) on the MACU 770. MACU 770 also preferably features an ECG electrode 714. ECG electrode 714 may be used as the third electrode for enabling the ECG measuring. Also, MACU 770 preferably features a SpO₂ sensor 716 as shown.

According to an embodiment of the present invention, MACU 770 also features a communication port 718 for enabling device 700 to communicate with an external electronic device such as a computer for example. Port 718 is optionally a serial port, such as a RS232 port for example, or any other suitable type of port.

MACU 770 also preferably features a sensor port 720 for receiving a connector from an external monitoring device (not shown), such as but not limited to a SpO2 monitoring device (not shown), which may for example fit on or over a finger of the subject. Sensor port 720 may be used for connecting other analog devices such as additional ECG electrode, etc.

FIG. 7C shows MACU 770 again from a different angle, in order to show an optional but preferred temperature sensor port 722 for receiving a connector from temperature sensor 712. Alternatively, temperature sensor 712 may communicate with MACU 770 through wireless communication (not shown).

FIG. 7D shows temperature sensor 712 alone.

FIG. 8 shows a schematic block diagram of a system according to the present invention featuring the blood pressure monitoring device of FIG. 7, wearable device 700. Device 700 preferably features a number of components, which were previously described in conjunction with FIGS. 1 to 7. Components having the same reference number have the same or similar function unless otherwise stated.

As part of an exemplary embodiment of the system of the present invention, wearable device 700 preferably features a movement sensor 810 which may be an accelerometer. Movement sensor 810 may be used for measuring breathing. According to an embodiment of the present invention, the user places wearable device 700 against the abdomen or chest and then breathes (inhales and exhales). The movement of the stomach or chest during breathing is measured by movement sensor 810 and is used to measure breathing, for example according to rate of respiration, whether breathing is shallow or deep, whether the user is coughing and/or experiencing other respiratory difficulty or distress, and so forth. Movement sensor 810 may measure movements along one axis or more than one axis.

Movement sensor 810 preferably communicates with processing unit 103 in order to provide a signal indicating the measurement(s) being taken for breathing (respiration); this signal is then preferably analyzed as previously described.

It should be noted that screen 701 of FIGS. 7B and 7C is shown here as display 124, which may optionally be an LCD for example. Screen 701 preferably is controlled by processing unit 103. Also as described in greater detail below, screen 701 preferably is capable of displaying an electronic medical calendar or other medical management function, for example to remind the user to take certain medication(s) at certain time(s), to remind the user to attend a medical appointment at a certain time and place, and/or to remind the user to be certain to perform one or more physiological measurements with the device according to the present invention. These functions may optionally and preferably be synchronized through communication with remote server 114, as described in greater detail with regard to FIG. 10.

Also as described with regard to FIG. 7A, wearable device 700 preferably features a unit for measuring blood pressure, shown here as a blood pressure unit 820 for measuring and/or monitoring the blood pressure of the user. Blood pressure unit 820 may optionally and preferably be implemented according to oscillometry (see www.colin-europe.com as of Feb. 10, 2004 for example). Oscillometry does not determine blood pressure as an instant measurement, but instead calculates blood pressure according to the curves of changes (derivatives) in blood pressure and its oscillation. An advantage of this method is that it is not affected by sources of external noise and/or the presence of other electronic devices in the area of the patient. Another advantage of this method is that even under severe hypotension (very low blood pressure) conditions, such that for example the Korotkoff sounds are barely detectable, oscillometry can still measure blood pressure as long as an arterial pulse exists. Typically, oscillometry is used while the pressure in the blood pressure cuff is first increased and then gradually decreased; as the cuff pressure decreases, the oscillation also decreases. The value of the systolic pressure is measured when the oscillation decreases rapidly, while the diastolic pressure value is measured when the oscillation decreases rapidly. The mean arterial pressure is measured when the oscillation reaches a peak. Blood pressure unit 820 may be divided into two sections, a control section that may be located at MACU 770 and an inflatable cuff that reside in the band of wearable device 700. The connection between the two sections may optionally be implemented with one or more air tubes (not shown).

Blood pressure monitoring unit 820 is an example of one type of sensor; preferably, as previously described, wearable device 700 features a plurality of sensors controlled through processing unit 103. An advantage of centralizing control through processing unit 103 is that measurements and activities of the different sensors may optionally be synchronized by processing unit 103, for example according to real time clock 117. Also, a comparative display of the measurements from different sensors may also optionally be provided to remote server 114 and/or display 124, preferably with synchronization between the timing of the measurements from the different sensors.

Preferably, a locator unit 830 is featured, in order to be able to locate wearable device 700, thereby also locating the user who is wearing wearable device 700. This feature is optional but preferred because the user may be a patient who is elderly or otherwise incapacitated, and who therefore may become lost or disoriented. Locator unit 830 preferably enables a caregiver or other individual to locate the patient. Optionally and preferably, locator unit 830 comprises a GPS unit, which could easily be implemented by one of ordinary skill in the art. Locator unit 830 preferably communicates with processing unit 103 in order to relay location information to remote server 114, if for example the subject cannot communicate this information. For the implementation with a GPS unit as shown, locator unit 830 also preferably features an antenna 834 as is well known in the art.

A user input interface 840 optionally and preferably enables the user to input information and/or commands. More preferably, user input interface 840 preferably communicates with at least one, but most preferably a plurality of buttons (see FIG. 7) for receiving information and/or commands from the user.

As described with regard to FIG. 7, wearable device 700 preferably also features a serial interface port 850, such as a RS232 port for example, for communicating with an electronic device, such as a glucometer (blood glucose meter) and/or a computer for example, and/or getting software updates.

According to an embodiment of the present invention, preferably communication unit 104 and/or 107 each independently comprises one or more different types of communicators; for example, communication unit 104 and/or 107 may optionally comprise one or more of wireless communication such as WiFi or cellular telephone communication, or relatively short range communication such as Bluetooth for example, all of which are known in the art and could easily be implemented by one of ordinary skill in the art. Communication may optionally be continuous or intermittent. Other embodiments may use proprietary protocols.

FIG. 9 is a flowchart of an exemplary method according to the present invention for performing an extended physiological monitoring task. The method is described with reference to a “Holter” task for the purpose of illustration only and without any intention of being limiting. As is known in the art, a Holter task refers to a type of cardiac monitoring (including but not limited to blood pressure, ECG monitoring) which is well known in the art, and which is typically performed over 24 hours on a (frequently) ambulatory patient, whether in a hospital or as an out-patient. Electrodes are placed on the chest of the patient and attached to a small heart-recording monitor that can be carried in a pocket or small pouch worn around the neck of the patient for example. The monitor is battery operated. The patient's heart activity is recorded, usually for a 24-hour period while the patient also maintains a manually written diary of activity during the 24-hour period. The recording is then analyzed by medical personnel, a report of the heart's activity is tabulated, and irregular heart activity is correlated with activity.

The present invention offers an advanced Holter method that may use the wearable device 700 (FIG. 7) as an improved Holter device. The present invention may add additional physiological parameters to the Holter task, including but not limited to SpO₂, blood pressure, body temperature, breathing rate, etc. The different physiological parameters may be measured in a synchronized manner by using the same time axis. Synchronizing the different measurements may improve the understanding of the different readings. The operation of the Holter equipment according to an embodiment of the present invention is simple, and user friendly, since the wearable device includes a blood pressure cuff and two electrodes for ECG measurements. The third electrode may be extended, by an additional cable, from the wearable device 700 (FIG. 7) and be placed over the subject's chest.

In addition the present invention may eliminate the need for a manually written diary of activity during the 24-hour period, as instead the subject may enter information about the type of activity that the subject is performing into the wearable device. Entering the type of activity may optionally be done by selecting the appropriate activity from a selection of activities that are displayed by display 701 through the user interface buttons 702, 706 and 704 (FIGS. 7B and 7C).

Furthermore the present invention may be implemented with an event recorder; a person may optionally instruct wearable device 700 to manually activate an event recording if symptoms such as palpitations, chest pain, or irregular rhythms are noticed. Then the wearable device may run a loop with a plurality of measuring for a short period of time, few minutes to tens of minutes and record the results.

Other embodiments of the present invention may have loop recorder capabilities. In loop recording, a time cycle is determined for a certain period of time, for example, ten minutes, twenty minutes, an hour, etc. During the loop a plurality of measuring cycles is performed. Each measuring cycle may be performed with measurements of one or more physiological parameters. The results of the measurements are recorded in a memory section that may be dedicated to the loop recording function. At the end of the loop period a next loop begins, and the next loop results are written over information from the previous loop. Therefore at every moment, the loop memory includes information from the last period, which is continuously overwritten to be current, as for the “black box” in an airplane.

At any given time a user may inform the wearable device that symptoms like palpitations, chest pain, etc. are currently felt. From that moment the loop is preferably broken, and the wearable device preferably continues the measurement(s) for a certain period, recording the results in addition to the information that was stored during the loop that was broken. At the end of the period, a loop recording may optionally be started again, but preferably featuring storage of the information in another loop section in the memory. This feature may be used by a doctor for reviewing the physiological parameters of the subject prior and during a particular event.

The present invention is able to perform a more easily documented and more convenient type of Holter monitoring as described with regard to FIG. 9. As shown, in method 900 (indicated by an arrow), monitoring is optionally initiated 910 by an external signal to the wearable device 700 (FIG. 7), a manual activation, or a signal from an internal electronic calendar that may be updated by the remote server according to an exemplary embodiment of the present invention. The signal may optionally be provided according to a medical management function such as a “calendar” for example, in which the time to start monitoring may optionally be entered. The signal may also optionally be provided according to a timer and/or according to a signal external to the device according to the present invention itself, such as from a call center for example.

Next, the physiological monitoring (Holter) task is preferably started in stage 910. The device then obtains the Holter parameters in stage 912, such as determining which types of measurements are to be performed, the length of the period in which the Holter task is performed, the Holter's period, and time interval between each measuring event that will be preformed during the Holter's period. Other parameters may define the event, and each event may use one or more sensors for measuring one or more physiological parameters. The present invention may use more than one type of measuring event during a Holter's period. These measurements may optionally include one or more measurements from the sensor(s), including but not limited to ECG, blood pressure, SpO₂, pulse rate, temperature, respiration (breathing) functionality and so forth.

In stage 914, the device optionally communicates with the patient (subject) in order for the patient to be aware that the Holter monitoring is to start. The response may optionally include performing a measurement by the user, for example of temperature. If no response is received, preferably the device again attempts to communicate with the patient until a response is received or a given number of attempts and/or elapsed time has passed, after which optionally an alarm is raised and the method exits. A user response optionally may not be required. In some cases, steps 914 and 920 may be added to each measuring event, between steps 930 and 932. Communicating with the subject may be needed in case that the subject's participation is required to perform a certain action. For example, the subject may be requested to place a finger over the SpO₂ sensor.

In stage 920, if the user has responded and/or if no response is required, then the measuring process starts, optionally separately for each measuring event in stage 930. A measuring event is preferably started with the appropriate sensors (stage 932), after which the results of the measurement(s) are stored, in memory 105, preferably with the time of measurement (stage 934). Different events, during the same Holter period, may use different sensors depending on the Holter's parameters. For example, ECG sensors may be used in each event (from every few seconds to a few minutes, for example), while the blood pressure or temperature may be measured in events that occur once an hour, for example. The event is optionally and preferably controlled according to the timing provided by the real time clock as previously described, such that synchronization is preferably possible as previously described and also for more accurate determination of the time of measurement. After each event ends, the timing for the next event is preferably determined (stage 936), preferably according to a predetermined program, as disclosed above in conjunction to step 912. The program may optionally be sent to the device from an external source, such as a call center and/or remote server for example.

Optionally at one or more points during the Holter monitoring period, the user enters a note about an activity that the user is performing, such as climbing stairs for example. These notes may then optionally and preferably be correlated with cardiac activity as previously described. Preferably, the user is able to enter the note to the device according to the present invention through a note-taking function, more preferably by selecting from a plurality of predetermined menu choice activities (such as climbing stairs, getting out of bed, defecating and so forth).

At the end of the waiting period 936 for the next measuring event, a decision is made 940 as to whether the entire Holter period is terminated. If not, then method 900 returns to step 930 and starts an additional measuring event. If the period is terminated, the measurement results are preferably then transmitted from the device, for example to the call center and/or remote server or other external location via gateway 110 (FIG. 8), in stage 942. Other exemplary embodiments may transfer the result of the measurements at the end of each event, instead of storing them in the wearable device (stage 934).

FIG. 10 is a flowchart of an exemplary method according to the present invention for synchronizing a medical care management function of the device according to the present invention with a central care facility. As for the method of FIG. 10, the process of synchronization may optionally be initiated by an internal or external signal to the device according to the present invention, as shown in method 1000 (indicated by an arrow).

In stage 1010, the medical diary (management function) synchronization task starts. T he management function determines whether an event is to occur in stage 1020. An event may be, for example, taking a medicine, starting to measure certain physiological parameters that require an active action from the subject (i.e. placing the thermometer under the subject's tongue), or transmitting text messages that were sent from the call center to the subject, such as the date of the next visit to a medical office, etc. If no event is to occur, then the process proceeds to establish a connection with an external synchronization source, such as a call center for example, in stage 1036.

If an event is to occur, then the event is communicated to the subject in stage 1022; for example the device may optionally make a sound, to catch the attention of the subject, such as a beep for example, in addition to which an instruction may be displayed over display (screen) 701 (FIG. 7a). In stage 1030, it is determined whether feedback (a response) from the subject is required. Feedback may be needed if the subject is requested to act, in order to indicate that the subject has fulfilled the instruction (for example, if the subject has been requested to place the thermometer under the subject's tongue). An exemplary feedback may be done by pressing button 708 (FIG. 7) after taking the medicine. If no feedback is required, then the process also proceeds to performing the event such as measuring the skin temperature, for example, in stage 1035.

If feedback is required, then optionally the process waits to receive the feedback, preferably for a time period T1 (in which T1 is the length of time which should elapse), in stage 1032. If feedback is then not received by the end of time period T1, then the process preferably returns to stage 1022 and the event is communicated to the subject again. Optionally and preferably, the process is only allowed to return to this point a predetermined number of times and/or for a predetermined period of time, to avoid holding in a loop; more preferably an alarm is raised once this threshold has passed.

If feedback is received, then the process 1000 may perform the event (measuring task) 1035 before establishing the connection with the call center. The measuring task may have optionally been previously described to the subject and/or given as a previous alert to the subject. Then method 1000 also proceeds (in stage 1036) to establish a connection with an external synchronization source, such as a call center for example. Once the connection has been established, the medical diary (management function) in the wearable device is synchronized in stage 1038. There are other exemplary synchronized methods in which the wearable device may communicate with the call center independently from method 1000. Communicating with the call center may be done at any other time than the synchronization task (method 1000), for example when the free space in the memory of the wearable device is below a certain level. In those methods, the synchronization between the medical diary in the wearable device and the relevant diary in the call center may be done during the next connection with the call center. The process then ends in stage 1040, for example optionally by ending the connection with the external source, or by entering to a sleeping mode until the time of the next synchronization cycle.

It will be appreciated that the above descriptions are intended only to serve as examples, and that many other embodiments are possible within the spirit and the scope of the present invention.

What is claimed is:

1. A device for measuring at least one physiological parameter of a subject in addition to blood pressure, comprising:
(a) a band for being fastened to a hand of the user;
(b) a blood pressure cuff for being associated with said band;
(c) at least one additional sensor for measuring at least one additional physiological parameter of the user; and
(d) a monitor and control unit (MACU) for controlling said blood pressure measurement and said measurement of said at least one additional physiological parameter of the user and for receiving a signal from said sensor and a signal from said blood pressure cuff and for converting said signals to form medical information.

2. The device of claim 1, wherein said physiological parameter includes breathing rate.

3. The device of claim 2, comprising a movement sensor for measuring the breathing rate.

4. The device of claim 1, wherein the at least one additional sensor includes an electro cardiogram (ECG) sensor and wherein the ECG sensor includes at least one electrode associated with said band.

5. The device of claim 4, wherein said band features at least a rigid portion and said at least one electrode is associated with said rigid portion.

6. The device of claim 4, wherein the at least one electrode is organized into a set of two or more electrodes, such that only one electrode is required to be in contact with said wrist at any particular time.

7. The device of claim 1, wherein said MACU further comprises a sensor port for communicating with an external monitoring device for monitoring a physiological parameter.

8. The device of claim 1, wherein said physiological parameter is oxygen saturation in the blood (SpO₂).

9. The device of claim 8, wherein said at least one sensor comprises an SpO₂ sensor.

10. The device of claim 1, wherein said physiological parameter includes body temperature.

11. The device of claim 10, wherein said at least one sensor comprises a temperature sensor.

12. The device of claim 1, further comprising:
a display mounted on said device.

13. The device of claim 12, wherein said display shows at least one of a measurement from said sensor or from said blood pressure cuff (or a combination), or a medical diary management information, or a user interface.

14. The device of claim 1, further comprising:
a non-volatile memory for storing at least one physiological measurement result.

15. The device of claim 1, further comprising:
a communication unit for at least transmitting data.

16. The device of claim 15, wherein said communication unit also transmits a device identifier for uniquely identifying the device.

17. The device of claim 15, wherein said communication unit also receives data.

18. The device of claim 17, wherein said data includes information for synchronizing a medical diary management function.

19. The device of any of claims 1-18, further comprising a locator unit for locating the device.

20. The device of claim 19, wherein said locator unit comprises a GPS unit.

21. A system for measuring at least one physiological parameter of a subject and also blood pressure, comprising:
(a) a device for measuring the at least one physiological parameter and blood pressure, comprising:
(i) a blood pressure cuff for being fastened to a wrist of the user;
(ii) at least one additional sensor for measuring at least one additional physiological parameter of the user;
(iii) a processor for receiving a signal from said sensor and a signal from said blood pressure cuff and for converting said signals to form data;
(iv) a communication unit for at least transmitting data; and
(b) a gateway device for receiving said transmitted data for being monitored.

22. The system of claim 21, further comprising:
(c) a remote server in communication with said gateway device, said remote server optionally being part of a call center for monitoring.

23. The system of claim 22, wherein said measurement of said at least one additional sensor is combined with blood pressure measurement to determine if said measurements are outside of an acceptable range, and if so, alerting said remote server.

24. A method for performing extended physiological monitoring for a Holter task on a subject for collecting results from a plurality of measuring events, with the device or system of any of claims 1-23, comprising:
initiating an extended physiological monitoring task with the device;
determining the Holter period by determining a time interval between each pair of measuring events;
determining at least one type of measuring event for said monitoring task;
performing at least one measuring event;
storing a result of said at least one measuring cycle; and
providing said result.

25. The method of claim 24, wherein at least one measuring event is performed for two or more physiological parameters.

26. The method of claims 24 or 25, wherein said result is transmitted to a remote server and/or call center.

27. The method of claim 26, wherein the results of the different physiological parameters are determined by reference to the same time base along the Holter period.

28. The method of claim 27, wherein the different physiological parameter includes one or more physiological parameters s elected from the group consisting of ECG, blood pressure, SpO2, temperature, and breathing rate.

29. The method of claim 27, wherein said initiating further comprises communicating with the subject.

30. The method of claim 27, wherein said initiating is determined according to at least one of an external signal from an external source or an internal timing signal.

31. The method of claim 30, wherein said internal timing signal is determined according to a medical diary management function.

32. The method of claim 30, wherein said initiating is determined according to a manual command from the subject.

33. The method of claim 30, wherein the subject enters at least one note about at least one activity of the subject during the Holter period.

34. The method of any of claims 30-33, implemented as a Holter monitoring process.

35. A method for measuring breathing rate of a subject with the device of any of claims 2-23, comprising:
placing the device against the stomach or chest of the subject;
measuring the movement of the device; and
determining the breathing rate of the subject.
